(19) United States
(12) Patent Application Publication
(10) Pub. No.: US 2021/0319377 A1
(43) Pub. Date: Oct. 14, 2021

(54) TEST METHOD FOR DETERMINING THE RISK POTENTIAL FOR AN ALKALI-SILICA REACTION IN MINERAL CONSTRUCTION MATERIALS

(71) Applicant: FRAUNHOFER-GESELLSCHAFT ZUR FÖRDERUNG DER ANGEWANDTEN FORSCHUNG E.V., Munich (DE)

(72) Inventors: SEBASTIAN DITTRICH, ROSENHEIM (DE); VOLKER THOME, SCHLIERSEE (DE); SEVERIN SEIFERT, RAUBLING (DE); MIRIAM KRÜGER, TAUFKIRCHEN (DE)

(73) Assignee: FRAUNHOFER-GESELLSCHAFT ZUR FÖRDERUNG DER ANGEWANDTEN FORSCHUNG E.V., MUNICH (DE)

(21) Appl. No.: 17/358,583

(22) Filed: Jun. 25, 2021

Related U.S. Application Data
(63) Continuation of application No. PCT/EP2019/086885, filed on Dec. 22, 2019.

(30) Foreign Application Priority Data
Dec. 28, 2018 (DE) 102018251789.4

(51) Int. Cl. G06Q 10/06; G01N 21/65; G01N 33/38; G06Q 50/08

(52) U.S. Cl. CPC G06Q 10/0635; G06Q 50/08; G01N 33/383; G01N 21/65

(57) ABSTRACT

The subject matter of the present application relates to a test method for determining the risk potential of an alkali-silica reaction in mineral construction materials such as concrete. The test method may be characterized by the steps of: a) examining (220) a sample (12) by means of Raman spectroscopy for providing a structural characterization (230) of the sample (12), b) comparing (240) the examination result to values (250) stored in a database, and c) using (260) the result of the comparison (240) for ascertaining (270) a risk potential for the concrete for an alkali-silica reaction.

### RELATED APPLICATIONS

This is a Bypass Continuation of International Application No. PCT/EP2019/086885 filed Dec. 22, 2019 and published as WO 2020/136152A1. Priority is claimed to DE 10 2018 251 789.4, filed Dec. 28, 2018. The contents of the above-identified applications are incorporated by reference in their entirety.

### FIELD OF THE INVENTION

The invention relates to a test method for determining the risk potential of an alkali-silica reaction (ASR) in mineral construction materials, such as concrete.

### BACKGROUND

Concrete is one of the most important structural construction materials worldwide. In general, concrete exhibits high durability which, in some cases, can however be reduced by a material-related damage reaction. Due to a reaction between the alkali-sensitive or -reactive SiO₂-rich aggregates and the alkalis and hydroxides from the pore solution in the concrete, an alkali-silica gel (ASR gel) is formed. The ASR gel swells due to the absorption of water, which in turn can lead to significant damage, such as cracking in the concrete or even spalling. The swelling pressures occurring when the ASR gel expands damage the texture in the concrete and reduce its service life. In Germany, for example, over 400 km of highway are affected by the effects of an alkali-silica reaction (ASR). ASR is a very slow process and damage usually occurs only after five years. The test methods currently used to determine whether there is a potential for an ASR are either lengthy or, due to a reduced test duration, do not provide reliable results. This is mainly due to the fact that the influence of the structural nature of both the starting materials (aggregate) and the reaction product (ASR gel) on an ASR is not sufficiently known.

In 1974, the Deutscher Ausschuss fur Stahlbeton e.V. (German Committee for Reinforced Concrete) introduced an alkali guideline entitled “Vorbeugende Maßnahmen gegen schädigende Alkalireaktion im Beton” (Preventive Measures against Damaging Alkali Reaction in Concrete) (DAfStb guideline) for the control or testing of concrete as a construction material with regard to an ASR. A current edition of the DAfStb guideline was published in October 2013.

Appendix B of the DAfStb guideline describes a rapid test method known as mortar testing. In addition, a long-term test method (concrete test) is described. Up till now, the assessment of the alkali sensitivity or reactivity of aggregates is carried out on the basis of a rapid test method (mortar test) and/or a long-term test method (concrete test). The mortar rapid test is based on the production of mortars according to a predetermined formulation and subsequent storage in alkali or over water at elevated temperature. The change in length/expansion of the test specimens is examined at predetermined time intervals, as described in conjunction with FIG. 10. However, the use of the mortar rapid test only allows an exact classification of aggregates that are insensitive to alkali. For a safe risk assessment of the alkali-sensitive aggregates with regard to an ASR potential, it is absolutely necessary to carry out further long-term test methods (concrete test).

Concrete tests at 40° C. fog chamber storage (at 100% room humidity) or as a 60° C. concrete test are carried out in accordance with the DAfStb guideline within a storage period of 9 months or 20 weeks. In this case, the change in length/expansion of the test specimens is measured, as a result of which it is possible to assess the employed aggregates in terms of their ASR risk.

The article “Entwicklung eines direkten Prüfverfahrens zur Alkaliempfindlichkeitsbeurteilung von Gesteinskörnungen—der BTU-SP-Schnelltest” (Development of a Direct Test Method for Assessing the Alkali Reactivity of Aggregates—the BTU-SP Rapid Test), Forum der Forschung 20/2007: pages 73-78, BTU Cottbus, self-publisher, ISSN no.: 0947-6989, describes another well-known test method, the BTU-SP rapid test of the BTU Cottbus. This rapid test, which is shown in FIG. 11, is a direct test method on aggregates to examine the alkali sensitivity. During a storage for 14 days in a KOH solution at elevated temperature, a portion of the solution is removed. Solution analyses are carried out, from which the excess silica is calculated. In addition to the silica excess, the open porosity of the aggregates is determined. The results are used to classify the alkali sensitivity. The problem with this method is that it is not sufficiently verified, so that the conclusions are uncertain.

Optimizations of known mortar and concrete test methods are described, for example, in the following patent applications:

JP 0273156 A discloses a test of an alkali aggregate for evaluating the alkali-silica reactivity of the aggregate by measuring changes in length of a mortar bar.

EP 2 397 848 A1 discloses an automatic measuring method and a device for the continuous expansion measurement on artificially weathered test specimens under simulated conditions of accelerated aging. The measurement method disclosed therein is adapted to detect the influence of the alkali-silica reaction provoking storage in concretes and the accompanying change in length of test specimens without an interruption of weathering in situ. JP 2008 230882 A discloses to use for concrete or mortar a fine aggregate that is found to be harmless when testing the alkali aggregate reaction according to the alkali-silica reactivity test methods according to JIS A 1145 and JIS A 1146.

WO 14 171902 A1 discloses a mortar bar tester and a test method in which the change in length of the alkali-silica reaction is observed that occurs on concrete samples which are used in the construction industry.

### SUMMARY

The known methods have the disadvantage that the predictions on the basis of measurement results obtained in the short term are very inaccurate or not correct. The object of the invention is therefore to provide a simple and fast method that can produce good predictions for the sensitivity of concrete to the alkali-silica reaction.

According to the invention, this object is achieved by a method of claim 1. Advantageous further developments of the invention are found in the subclaims.

According to one embodiment of the invention, a test method for determining the risk potential for an alkali-silica reaction in mineral construction materials, such as concrete, is provided, said method comprising the following steps:
(a) examining a sample by Raman spectroscopy to structurally characterize the sample,
b) comparing the result of the examination with the values stored in a database, and
c) using the result of the comparison for determining a risk potential for the concrete for an alkali-silica reaction.

A major advantage of the method according to the present invention over known methods is that the method according to the invention requires little effort and that the result of whether or not there is an ASR potential for the examined aggregate is available after only a few minutes.

None of the known test methods deals with the examination of the structural nature of the reaction product (ASR gel) or the employed starting materials (aggregate) to classify the alkali sensitivity of aggregates. However, the crystallinity of the aggregates has a significant influence on the silicate solubility and thus on the damage potential of an ASR. In turn, the structure or the chemical composition of the ASR gels has a major influence on the swelling behavior of the gels and can therefore additionally be used to classify the alkali sensitivity of aggregates.

According to the invention, the method can comprise the step of storing values in a database for comparing the examination results to the values stored in the database.

According to the invention, the sample to be examined can be or comprise a starting material of the concrete mixture for the preparation of the concrete.

According to the invention, the starting material can be or comprise an aggregate.

According to the invention, the sample to be examined can be or comprise a reaction product forming in the concrete. In this regard, the reaction product can be or comprise an alkali-silica gel (ASR gel).

According to the invention, the sample to be examined can be subjected to a dissolution test prior to the examination.

In this regard, the dissolution test can be carried out for at least 1 week. In this regard, the dissolution test can be carried out for at least 2 weeks. In this regard, the dissolution test can be carried out for at least 3 weeks.

In this regard, the dissolution test can be carried out at a temperature of more than 60° C. In this regard, the dissolution test can be carried out at a temperature of more than 70° C. In this regard, the dissolution test can be carried out at a temperature of more than 75° C. In this regard, the dissolution test can be carried out at a temperature of about 80° C. In this regard, the dissolution test can be carried out at a temperature of less than 95° C. In this regard, the dissolution test can be carried out at a temperature of less than 90° C. In this regard, the dissolution test can be carried out at a temperature of less than 85° C.

Depending on the temperature of the dissolution test, the duration can vary. For example, at a temperature of 80° C., a duration of 2 weeks can be selected.

According to the invention, the dissolution product can be or comprise an ASR gel, which is characterized by means of Raman spectroscopy to classify the risk potential of the sample.

According to the invention, the solvent can be or comprise K/NaOH. In this regard, the solvent can be or comprise 1 mol of K/NaOH.

According to the invention, portlandite Ca(OH)₂ can be added to the K/NaOH solution. Alternatively, the K/NaOH solution can be available without the addition of portlandite.

According to the invention, the Raman spectroscopy can be used as an ASR test method to classify the alkali sensitivity of aggregates. The ASR testing can be carried out on the basis of the structural examination of the starting materials (aggregate) and/or the resulting reaction products (ASR gels) in the concrete by means of Raman spectroscopy. Due to the use of the Raman spectroscopy it is possible to measure and structurally characterize, on the one hand, amorphous ASR gels present in mortar samples, in concrete samples or in solution and, on the other hand, also amorphous to crystalline aggregates. In order to classify the ASR damage potential, the measured Raman spectra are assessed by comparing these spectra with a previously created dedicated database.

The use of the Raman spectroscopy can provide new, important insights into the ASR and improve the assessment of an ASR risk. It can also provide another preventive measure against the ASR. This can increase the service life of concrete and save raw materials. Due to the small sample size and the X-ray amorphous structure of ASR gels, the Raman spectroscopy is currently the only measurement method to fully characterize the structure of ASR gels in a time-efficient manner. Therefore, the examination of the structure of the gels and the aggregates is an essential addition to the test methods used thus far.

ASR testing laboratories already accredited can use this method as a preventive measure. Furthermore, this invention might be used in the construction materials industry, such as in construction companies and raw material suppliers (e.g. gravel plants). Due to the use of Raman spectroscopy by means of a database, a faster and more efficient and simplified method for testing the ASR risk is possible. The use of this test method according to the invention cannot only fill some of the previous knowledge gaps on the ASR, but also improve the energy balance and allow the saving of raw materials, and altogether extend the service life of concrete.

The Raman spectroscopy can be used to purely characterize ASR gels or aggregates and not be used as a preventive measure/test method. This information can be used to indirectly infer the ASR damage potential of aggregates, for example.

### BRIEF DESCRIPTION OF THE DRAWINGS

Particular embodiments of the present invention are explained in more detail below with reference to the accompanying drawings:

FIG. 1 shows, according to an exemplary embodiment of the invention, a Raman spectrum of the reaction product/gel from a dissolution test of borosilicate glass.

FIG. 2 shows the assessment of the vibrational bands of the Raman shift at 585 cm⁻¹ of the Raman spectrum of FIG. 1 in region II.

FIG. 3 shows the assessment of the vibrational bands of the Raman shift at 1038 cm⁻¹ of the Raman spectrum of FIG. 1 in region III.

FIG. 4 shows, according to an exemplary embodiment of the invention, a Raman spectrum of a synthesized potassium-silicate gel.

FIG. 5 shows the assessment of the vibrational bands of the Raman shift at 530 cm⁻¹ of the Raman spectrum of FIG. 4 in region V.

FIG. 6 shows the assessment of the vibrational bands of the Raman shift at 1040 cm⁻¹ of the Raman spectrum of FIG. 4 in region VI.

FIG. 7 shows, according to exemplary embodiments of the invention, Raman spectra of various mineral grains of the examined aggregates using the example of graywacke.

FIG. 8 shows, according to exemplary embodiments of the invention, a Raman spectrum of various mineral grains of the examined aggregates using the example of borosilicate glass.

FIG. 9 shows, according to exemplary embodiments of the invention, Raman spectra of various mineral grains of the examined aggregates using the example of opal & flint sandstone.

FIG. 10 shows the schematic sequence of a known test method.

FIG. 11 shows the schematic sequence of a known test method.

FIG. 12 shows the schematic sequence of a test method according to the invention.

### DETAILED DESCRIPTION

According to the invention, Raman spectroscopy is used as an ASR test method to classify the alkali sensitivity of aggregates. The ASR testing is carried out on the basis of the structural examination of the starting materials (aggregates) and/or the resulting reaction products (ASR gels) in the concrete by means of Raman spectroscopy. By using Raman spectroscopy, both amorphous ASR gels can be present in mortar and concrete samples or in solution and amorphous to crystalline aggregates can be measured and structurally characterized. In order to classify the ASR damage potential, the measured Raman spectra is assessed on the basis of a comparison between these spectra and a previously created, dedicated database.

### Exemplary Embodiment 1

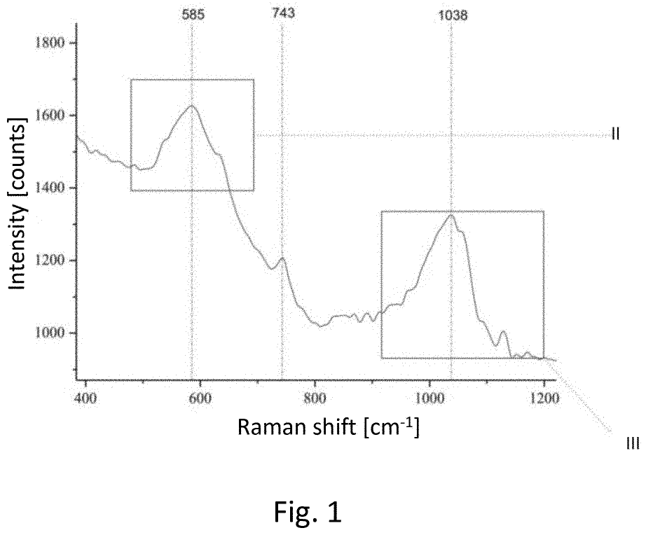

A dissolution test of fine-grained aggregates was carried out. Subsequently, the solution product (ASR gel) was characterized by means of Raman spectroscopy, as shown in FIGS. 1 to 3.

The dissolution test comprises the storage of fine-grained aggregates for at least 14 days in 1 mol K/NaOH solution with the addition of portlandite Ca(OH)2 at 80° C. Alternatively, the dissolution test can also be carried out without the addition of portlandite.

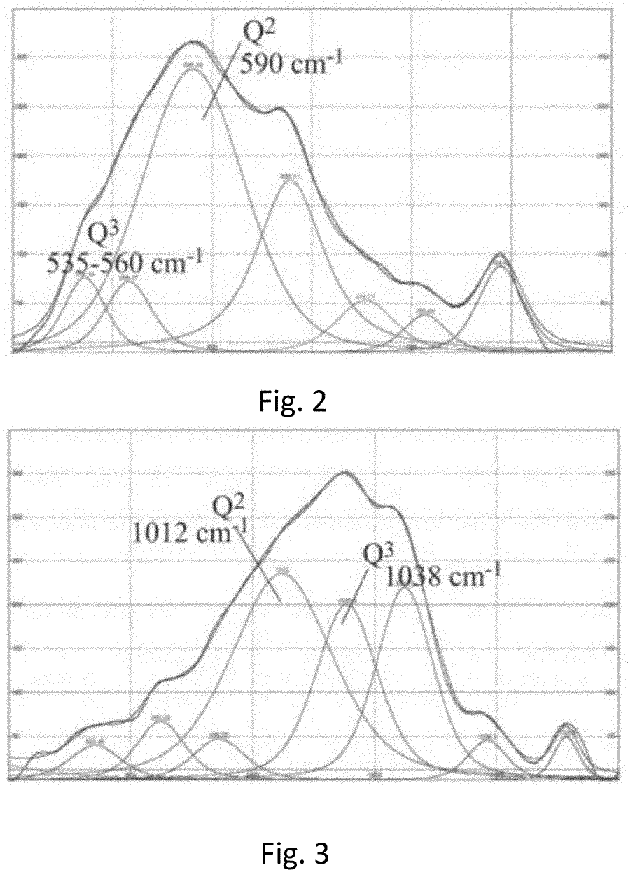

On the basis of an assessment of the vibrational spectra shown in FIGS. 2 and 3, it is possible to infer the alkali/Si ratio and the structure of the present gel and thus the alkali sensitivity of the aggregate.

The Raman spectrum shown in FIGS. 1 to 3 presents the result of a dissolution test of borosilicate glass.

The assessment of the vibrational bands according to FIGS. 2 and 3 allows the silicate linkages to be assigned and therefore provides information about the structure and/or properties of the ASR gel. A high degree of silicate linkage (Q4) here correlates with a high ASR resistance.

The determined image of the vibrational bands is compared with a database which records the correlations between the images of the vibrational bands and the ASR resistance from previously conducted long-term tests. The measure of ASR resistance was here determined by means of the above-mentioned known methods. The database thus makes it possible to link the ASR resistance determined in long-term tests with the rapid test methods for determining the images of the vibrational bands by means of Raman spectroscopy.

According to the invention, a structural examination of the starting materials of concrete that are subjected to a dissolution test is therefore carried out to classify the ASR risk.

This exemplary embodiment thus shows that the ASR risk potential can be determined with the help of the classification of the alkali sensitivity of aggregates. On the basis of the examination of the raw materials (aggregates), it is furthermore possible to determine an ASR risk potential with the help of the classification of the alkali sensitivity of aggregates.

### Exemplary Embodiment 2

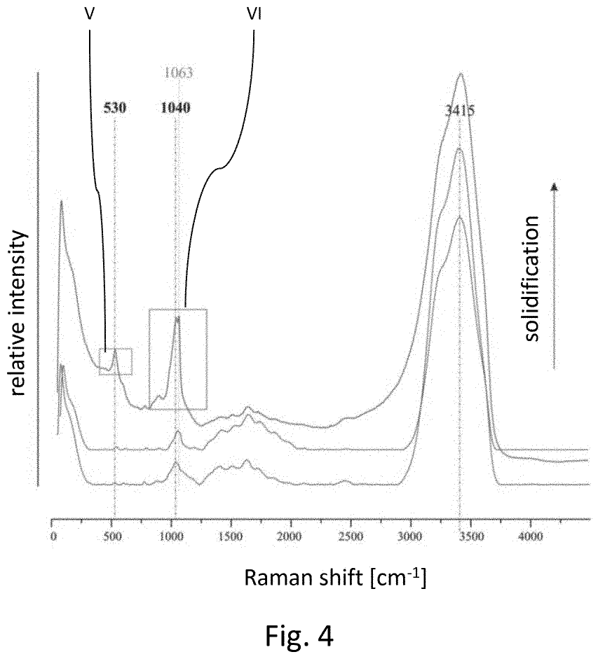

FIGS. 4 to 6 show a Raman spectroscopy of a synthesized potassium-silicate gel as it might occur in mortar/concrete samples.

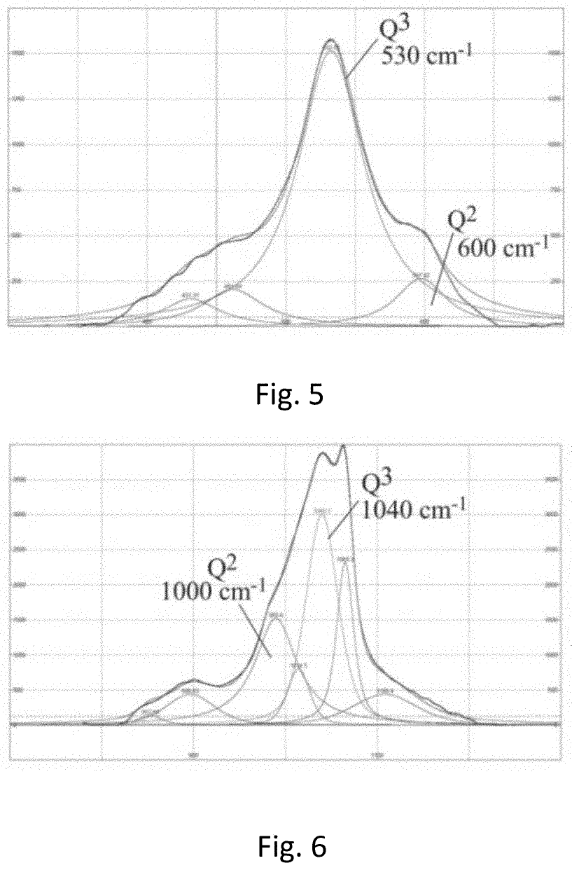

The gel was examined by means of Raman spectroscopy and structurally characterized on the basis of the vibrational bands shown in FIGS. 5 and 6. Due to the determined composition and structure, the ASR risk can be determined by comparison with a previously created database. It should be taken into account that the maximum swelling capacity of concrete depends on the composition and structure of the gel.

Instead of the synthesized gel, it is possible, according to the invention, to obtain the gel directly from a concrete sample.

This exemplary embodiment thus shows that the Raman spectroscopy according to the method of the invention can be used to detect and structurally characterize ASR gels. An ASR risk potential can be determined by a comparison with a database.

### Exemplary Embodiment 3

According to this exemplary embodiment, the aggregate is characterized with regard to structure by means of Raman spectroscopy. In other words, it is possible according to the invention to directly examine and characterize the aggregate by means of Raman spectroscopy without a prior dissolution test. The result of the characterization is then correlated with existing test methods (mortar/concrete test methods) by carrying out a comparison with a previously created database.

Various points of the aggregate are examined by Raman spectroscopy and the resulting spectrum is used to determine the structure and, obtained from this, the ASR risk. Depending on the spectral assessment of the measured minerals, a distinction can here be made between slow and fast reactive aggregates. If the structure of the SiO2 minerals is cryptocrystalline, lattice-disrupted or amorphous, a fast-reactive aggregate with a high risk potential is concerned. Crystalline SiO₂ minerals are either slowly reactive or have no ACR risk. The transition range between aggregates that have an ASR risk and those that have no ACR-risk is determined by a correlation with existing test methods.

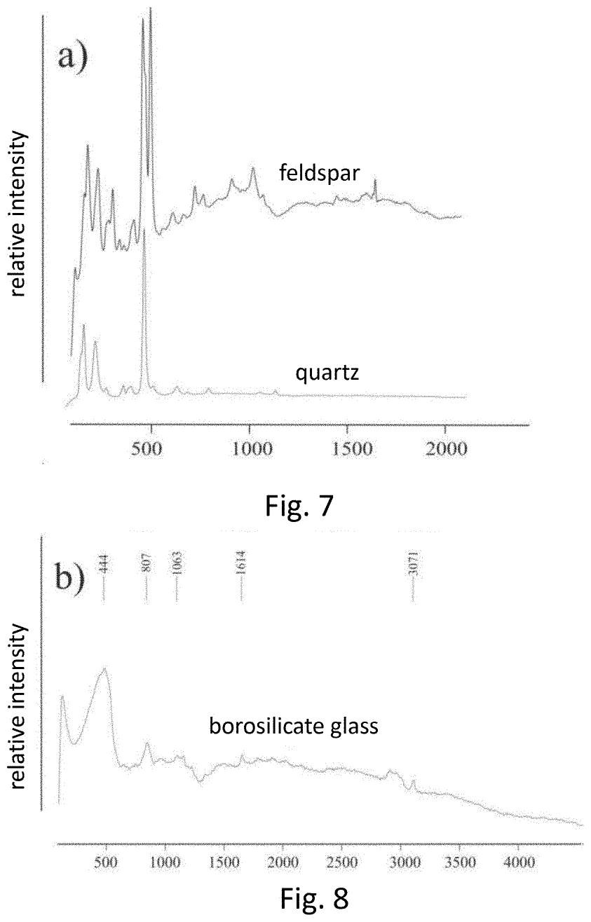

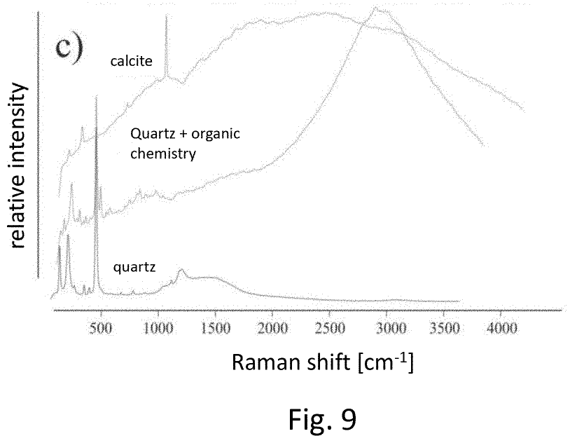

FIG. 7 shows a Raman spectrum of a slowly reactive aggregate (graywacke). FIG. 8 shows a Raman spectrum of a fast reactive synthetic aggregate (borosilicate glass). A comparison of the Raman spectra in FIG. 7 and FIG. 8 shows that the SiO₂ minerals of the slowly reactive aggregate (FIG. 7) are crystalline and the rapidly reactive aggregate (FIG. 8) is again amorphous.

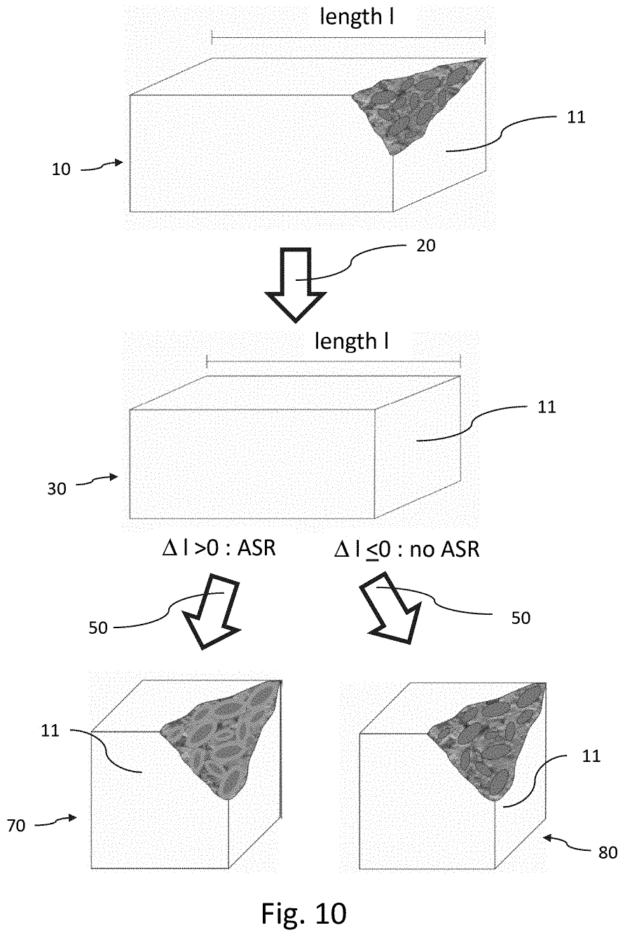

FIGS. 10 to 12 show the known methods in comparison with the method according to the invention.

FIG. 10 shows the method according to the DAfStb guideline. In step 10, a concrete sample 11 is shown and its length l is measured. In step 20, the concrete sample is stored for 9 months at 40° C. and 100% relative humidity. Alternatively, it can be stored at 60° C. for 20 weeks. In step 30, the concrete sample is measured again. In step 50, the change in length is used to determine the ASR risk potential. If length l decreases or remains the same, there is no ASR risk potential. If length l increases, there is an ASR risk potential. In steps 70 and 80, the assessment of the

ASR risk potential can be carried out in some circumstances with microscopic examinations of fresh cut surfaces of the test specimens 11.

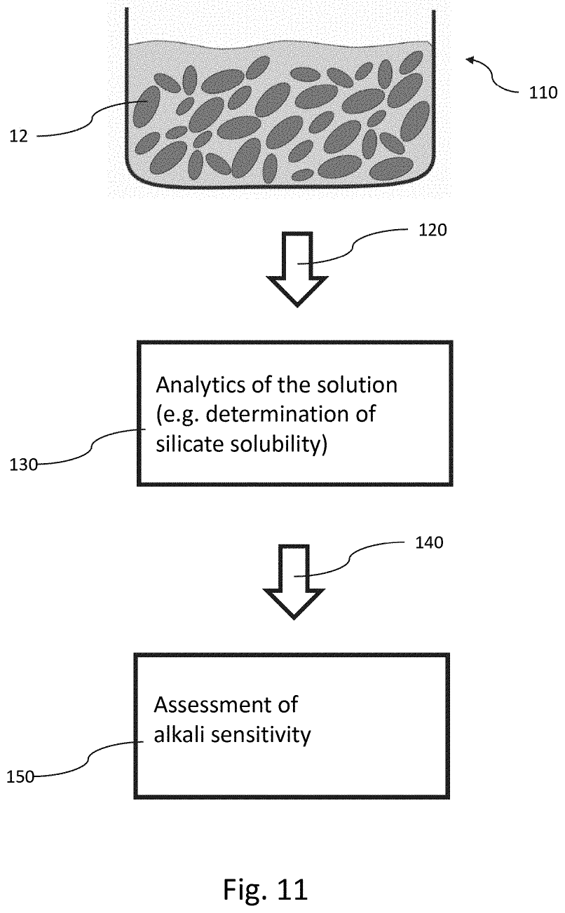

FIG. 11 illustrates the method according to the BTU-SP rapid test. In step 110, a gel 12 is provided as a sample. In step 120, the gel is stored for 14 days at pH14 and elevated temperature. In step 130, the solution is analyzed by determining the silicate solubility. In steps 140 and 150, the result provides an assessment of the alkali reactivity, which is a measure of the ASR risk potential.

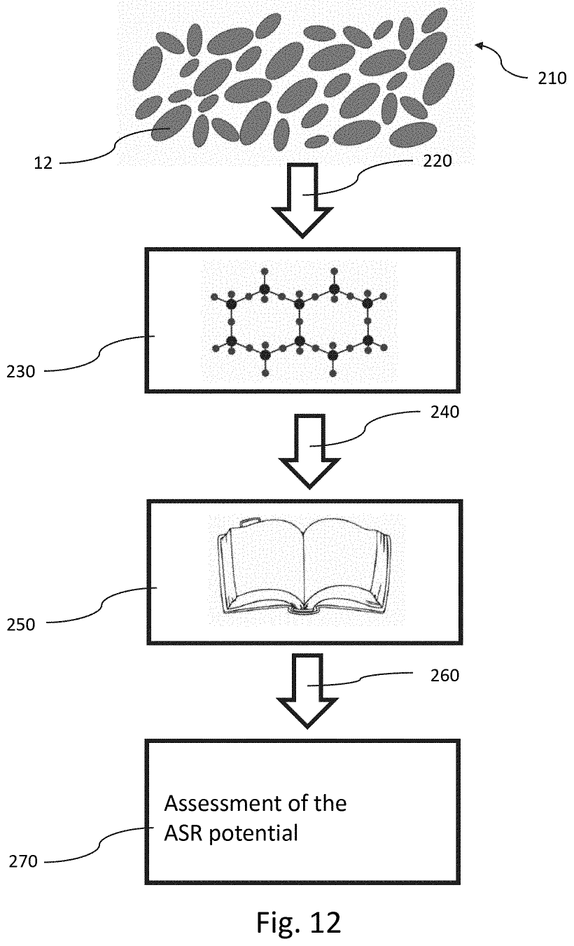

FIG. 12 illustrates the method according to the invention. In step 210, a sample 12 is provided. The sample 12 can be an aggregate or a gel. In step 220, an examination is carried out by means of Raman spectroscopy. Typically, the measurement can be performed in about 10 seconds. The measurement results can then be available almost immediately. For the time being, the assessment time is about 2 minutes. A structural analysis is carried out in step 230, and the results are compared with a database in steps 240, 250 and 260. Optionally, a correlation with the silicate solubility can be made according to the results from the methods shown in FIG. 11. The ASR risk potential is obtained from the comparison with the database.

A great advantage of the method according to the invention compared to the known methods is that the method according to the invention requires little effort and that the result of whether or not there is an ASR potential of the examined aggregate, can be available after only a few minutes.

Of course, the invention is not limited to the illustrated embodiments. Therefore, the above description should not be regarded as restrictive but as explanatory. The following claims are to be understood in such a way that a stated feature is present in at least one embodiment of the invention. This does not exclude the presence of further features. If the claims and the above description define “first” and “second” embodiments, this designation is used to distinguish between two similar embodiments without determining a ranking order.

What is claimed is:

1. Test method for determining the risk potential for an alkali-silica reaction in mineral construction materials, such as concrete, characterized by the following steps:
a) examining (220) a sample (12) by means of Raman spectroscopy for the structural characterization (230) of the sample (12),
b) comparing (240) the result of the examination with the values (250) stored in a database, and
c) using (260) the result of the comparison (240) for determining (270) a risk potential for the concrete with regard to an alkali-silica reaction.

2. Method according to claim 1, characterized in that the sample comprises a starting material of the concrete mixture for the preparation of the concrete.

3. Method according to claim 2, characterized in that the starting material comprises an aggregate.

4. Method according to claim 1, characterized in that the sample comprises a reaction product forming in the concrete.

5. Method according to claim 4, characterized in that the reaction product comprises an alkali-silica gel (ASR gel).

6. Method according to claim 1, characterized in that the sample is subjected to a dissolution test before being examined.

7. Method according to claim 6, characterized in that the solution product from the dissolution test comprises an ASR gel which is characterized by means of Raman spectroscopy in order to classify the risk potential of the sample.

8. Method according to claim 7, characterized in that the solvent comprises K/NaOH.

9. Method according to claim 8, characterized in that portlandite Ca(OH)₂ is added to the K/NaOH solution.

10. Method according to claim 6, characterized in that:
the solvent comprises K/NaOH; and
portlandite Ca(OH)₂ is added to the K/NaOH solution.

11. A method for determining risk potential for an alkali-silica reaction in concrete, comprising:
a) obtaining a structural characterization (230) of a concrete sample (12) using Raman spectroscopy;
b) comparing (240) the structural characterization (230) with values (250) stored in a database, to obtain one or more comparison results; and
c) determining (270) an alkali-silica reaction risk potential for the concrete, based on the comparison results.

12. The method according to claim 11, wherein the concrete sample comprises a starting material of a concrete mixture for the preparation of the concrete.

13. The method according to claim 12, wherein the starting material comprises an aggregate.

14. The method according to claim 11, wherein the concrete sample comprises an alkali-silica gel (ASR gel).

15. The method according to claim 11, comprising:
subjecting the concrete sample to a dissolution test with a dissolution solvent, prior to the step of obtaining a structural characterization.

16. The method according to claim 15, comprising:
producing an ASR gel during the dissolution test; and
characterizing the ASR gel by means of Raman spectroscopy in order to classify the risk potential of the concrete sample.

17. The method according to claim 16, wherein the dissolution solvent comprises K/NaOH.

18. The method according to claim 17, comprising:
adding portlandite Ca(OH)₂ to the dissolution solvent.

19. The method according to claim 15, wherein the dissolution solvent comprises K/NaOH.

20. The method according to claim 19, comprising:
adding portlandite Ca(OH)₂ to the dissolution solvent.
